(19) United States
(12) Patent Application Publication
(10) Pub. No.: US 2016/0250432 A1
(43) Pub. Date: Sep. 1, 2016

(54) METHOD AND APPARATUS FOR MULTI-CAMERA INTUBATION

(71) Applicants: Kumudhini HENDRIX, Bethesda, MD (US); Steven PRICE, Washington, DC (US)

(72) Inventors: Kumudhini HENDRIX, Bethesda, MD (US); Steven PRICE, Washington, DC (US)

(21) Appl. No.: 15/028,021

(22) PCT Filed: Oct. 1, 2014
(86) PCT No.: PCT/US14/58704

§ 371 Date: Apr. 7, 2016

Related U.S. Application Data
(60) Provisional application No. 61/889,524, filed on Oct. 10, 2013.

(51) Int. Cl. A61M 16/04; A61B 1/267; A61B 1/06; A61B 1/05

(52) U.S. Cl. CPC A61M 16/0488; A61B 1/051; A61B 1/267; A61B 1/0676; A61M 16/0429

(57) ABSTRACT

Exemplary embodiments of the present invention disclose a multi-camera intubation device for video-guided intubation. Video-guided intubation enhances the safety and success of intubation in medical practice by allowing practitioners to observe obstructions during intubation techniques, such as oropharygolaryngoscopy. A first camera supports visualization of the glottis while minimizing neck hyperextension and patient stimulation; a second camera vastly decreases the incidence of injury to the soft palate, palatopharyngeal arch, palatoglossal arch, and tonsil during passage of a rigid tube, such as a styleted endotracheal tube (ETT). This second camera would provide a real-time view of a patient's internal structures, for example, pharyngeal inlet, and thereby guide a safe, atraumatic intubation.

### CROSS-REFERENCE TO RELATED APPLICATION

This application claims the benefit of U.S. Provisional Application No. 61/889,524, filed Oct. 10, 2013, which is hereby incorporated by reference in its entirety

### TECHNICAL FIELD

The present disclosure relates to medical devices and, more particularly, to video-enabled intubation and oropharyngolaryngoscopy.

### BACKGROUND

The practice of clinical medicine has been aided with the implementation of high-resolution micro cameras that project real-time images that would otherwise not be practically visible. Various devices have been designed with the inclusion of a camera to facilitate use, including but not limited to the endoscope for evaluation of the gastrointestinal tract, the laparoscope to make abdominal surgery less invasive, and the videolaryngoscope to facilitate the placement of a breathing tube. The use of unidirectional, single camera technology as currently designed, however, is limited in scope and often dangerous, as nearby and or surrounding friable anatomic structures can be difficult or impossible to visualize. Hence, manipulation of these devices near such structures can lead to damage. Trauma of this sort is avoidable with proper visualization.

For example, direct laryngoscopy refers to the placement of a laryngoscope blade into a patient's mouth in order to expose the glottis and facilitate successful intubation (placement of a breathing tube, otherwise known as an endotracheal tube [ETT]) under direct visualization. Typically, a rigid stylet is inserted into the ETT prior to attempted intubation to facilitate its proper placement. Several advanced airway devices exist to aid in cases of failure or contraindication of direct laryngoscopy, notably the videolaryngoscope (VL). VL utilize a high-resolution micro camera at the end of a rigid laryngoscope to allow a line of sight to visualize glottic structures, such as the vocal cords, through which the ETT will be passed. Furthermore, this mode of intubation allows for neck neutrality, decreased patient stimulation, and improved ease of intubation.

The use of a VL, however, is not without risk. Multiple reports have been published that illustrate trauma to the oropharyngeal structures (including the soft palate, tonsil, palatopharyngeal arch, palatoglossal arch) caused by force placed on these structures during the passage of a rigid, styleted ETT. The mucosa in the pharynx is vascular and easily traumatized with minimal force. Additionally, important vascular and neural structures reside in the oropharyngeal cavity. Once traumatized, the oral structures easily swell and obscure further video or direct laryngoscopy. This may ultimately lead to failure to secure an airway, a subsequent decline in oxygenation, and eventual death.

In order to decrease risk of injury to oropharyngeal structures, manuals for the commonly used VLs instruct users to directly visualize the oropharynx as the styleted ETT is being passed. This approach is impractical, however, for at least two distinct reasons. First, it is unsafe to lose visualization of the glottis during attempted intubation. By diverting attention and focus from maintenance of a good glottic view, one can easily lose visualization of the glottis since even small movements by the operator can obscure glottic views. Often, once lost, adequate views that were carefully obtained can be difficult or impossible to recreate. Secondly, there is tremendous variability in the dimensions of pharyngeal structures amongst patients. Anecdotal experience has demonstrated that in many healthy patients, the tip of the ETT cannot be seen by direct visualization while it is advanced through the oropharynx towards the glottis. Furthermore, patients who require the use of VL intubation are often those with risk factors that will worsen the likelihood of successful intubation and eliminate the visibility of oropharyngeal structures during attempted intubation. Due to the current design of the VL, these inherent “blind spots” present regions of the oropharynx that are high risk for injury during VL.

### SUMMARY OF THE INVENTION

According to an embodiment of the present invention, a multi-camera intubation device is disclosed. The device includes a blade having distal and proximal ends separated by an elongated body, and a first camera located on the elongated body a distance from the distal end of the blade and providing a unidirectional, forward field of view. The device also includes a second camera located on the elongated body a distance proximal to the first camera and providing a field of view substantially orthogonal to the field of view of the first camera. In exemplary embodiments the distance of the second camera from the first can be adjustable.

According to an embodiment of the present invention, a multi-camera intubation device is disclosed. The device includes a blade having distal and proximal ends separated by an elongated body forming a radius adapted to conform to a contour of an oropharynx. The device further includes a first camera located on the elongated body a distance from the distal end of the blade and providing a unidirectional, forward, laryngeal field of view. The device further includes a second camera located on the elongated body a distance proximal to the first camera and providing a pharyngeal field of view substantially orthogonal to the field of view of the first camera to visualize a pharyngeal structure. In exemplary embodiments the distance of the second camera from the first can be adjustable.

According to another embodiment of the present invention, a method of intubating using a multi-camera intubation device is disclosed. The method includes the step of inserting a blade into a body cavity, the blade having distal and proximal ends separated by an elongated body with first and second cameras located thereon. The first camera is located a predetermined distance from the distal end of the blade and provides a unidirectional, forward field of view. The second camera is located proximal to the first camera and providing a field of view that is substantially orthogonal to the field of view of said first camera. The method also includes the step of viewing, on a display, a video output of the second camera to detect tissue obstructions. The method further includes, guided by the blade, the step of inserting a tube into the body cavity in a manner that minimizes trauma to the tissue obstructions detected by the second camera. In exemplary embodiments the tube comprises a rigid stylet.

Other objects, features, and advantages will be apparent to persons of ordinary skill in the art from the following detailed description and the accompanying drawings.

### BRIEF DESCRIPTION OF THE DRAWINGS

The above and/or other exemplary features and advantages of the preferred embodiments of the present disclosure will become more apparent through the detailed description of exemplary embodiments thereof with reference to the accompanying drawings, in which:

FIG. 1 depicts an exemplary embodiment of a multi-camera intubation device in accordance with the present invention;

FIG. 2 depicts a top view of an exemplary embodiment of a multi-camera intubation device in accordance with the present invention;

FIG. 3A depicts a perspective view of an exemplary embodiment of a multi-camera intubation device in accordance with the present invention;

FIG. 3B depicts a perspective view of an exemplary embodiment of a multi-camera intubation device in accordance with the present invention;

FIG. 4A depicts a perspective view of an exemplary embodiment of a multi-camera intubation device in accordance with the present invention illustrating orthogonal fields of view;

FIG. 4B depicts a perspective view of an exemplary embodiment of a multi-camera intubation device in accordance with the present invention illustrating orthogonal fields of view; and

FIG. 5 is a flow chart showing a multi-camera intubation process in accordance with an exemplary embodiment of the present invention.

Throughout the drawings, like reference numbers and labels should be understood to refer to like elements, features, and structures.

### DETAILED DESCRIPTION

Exemplary embodiments of the present disclosure will now be described more fully with reference to the accompanying drawings. The matters exemplified in this description are provided to assist in a comprehensive understanding of various embodiments disclosed with reference to the accompanying figures. Accordingly, those of ordinary skill in the art will recognize that various changes and modifications of the embodiments described herein can be made without departing from the scope and spirit of the claimed inventions. Descriptions of well-known functions and constructions are omitted for clarity and conciseness. To aid in clarity of description, the terms “upper,” “lower,” “above,” “below,” “left” and “right,” as used herein, provide reference with respect to orientation of the accompanying drawings and are not intended to be limiting.

Exemplary embodiments of the present invention introduce a multi-camera intubation device, for example, a video oropharyngolaryngoscope (VOPL). A VOPL in accordance with present embodiments comprise two cameras: one to present a view of the glottis similar to that which is currently in practice with VL to facilitate successful intubation; the second lateral camera would provide a panoramic view of oropharyngeal structures during passage of an ETT. This additional camera can be located on the side of the VOPL blade at the level of the soft palate, palatopharyngeal arch, palatoglossal arch and tonsil. Exemplary embodiments locate the second camera on the right side because laryngoscopes are customarily designed such that the ETT is passed through the right side of the mouth. It would provide a view of approximately 5 cm length by 4 cm width of oropharyngeal structures that would otherwise remain unseen. This second camera, which can utilize a wide-angle lens, can provide a panoramic image that will be transmitted to a section of the VOPL monitor. The camera can have a non-fog, wide-angle lens so that a lateral, wide view of the soft palatine, glossal, and tonsillar structures are fully visualized. As the styleted ETT is inserted, the tip of the ETT will be easily visualized in the display in real time as it passes through the pharynx, towards the glottis. With the oropharyngeal structures in view, the ETT can be safely advanced. The trauma to these structures during intubation will thus be avoided. In addition, the operator can gain important knowledge of the oropharyngeal structures and oral cavity of each patient. VOPL fundamentally transforms the design, functionality and safety of VL.

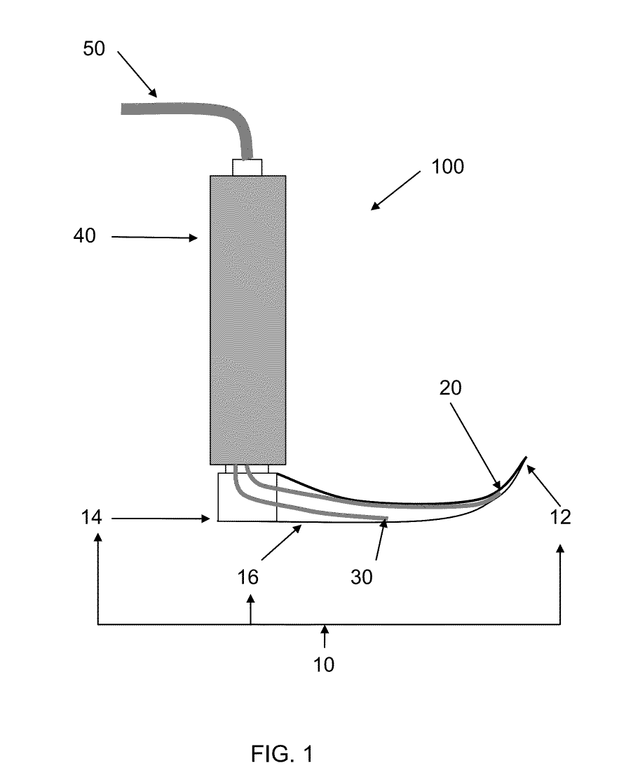

Referring now to FIG. 1, a multi-camera intubating device 100 is disclosed. The intubating device 100 comprises a blade 10 having distal 12 and proximal 14 ends separated by an elongated body 16. The intubating device 100 further includes a first camera 20 located on the elongated body 16 a predetermined distance from the distal end 12 of the blade 10 and providing a unidirectional, forward field of view. The device 100 further includes a second camera 30 located on the elongated body 16 a predetermined, adjustable distance proximal to the first camera 20 and providing a field of view that is substantially orthogonal to the field of view of the first camera 20. In exemplary embodiments the second camera 30 can provide a wide-angle field of view. In other embodiments the second camera 30 can provide panoramic fields of view. Exemplary embodiments provide a third camera located on the elongated body 16 in the vicinity of the second camera 30 and providing a field of view orthogonal to the field of view of the first camera 20 and opposite of the field of view of the second camera 30. A handle 40 can be attached to the proximal end 14 of the blade 10 to facilitate gripping by an operator. In an exemplary embodiment, the blade 10 would be placed into the mouth of a subject and advanced through the oropharynx until the distal end 12 abuts the vallecula in the oral cavity. User manipulation of the intubating device 100 at this point allows for the retraction of the epiglottis and thereby allowing the first camera 20 a view of the glottic opening. The second camera 30 allows for an orthogonal view within the oropharynx of vital pharyngeal structures including the tonsillar pillars, soft palate, palatopharyngeal arch, and palatoglossal arch. Visualization of these structures is vital to ensure an atraumatic passage of the styleted ETT through the oropharynx during attempted endotracheal intubation. In exemplary embodiments the blade 10 forms a radius to facilitate neck neutrality, decreased patient stimulation, and improved ease of intubation. In exemplary embodiments one or more light sources coupled to the blade 10 can be provided for illumination. Views from the first 20 and second 30 cameras can be visible on a display, as would be known to persons of ordinary skill in the art.

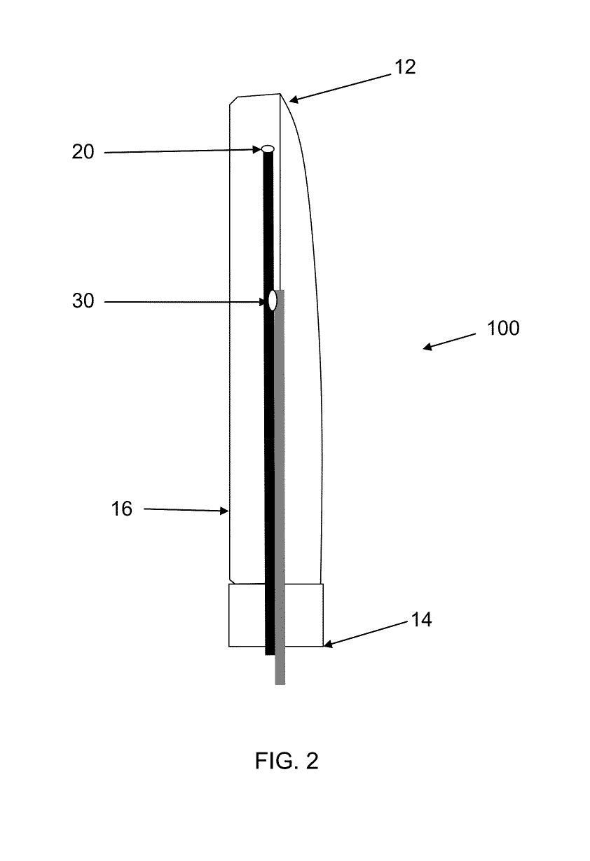

FIG. 2 depicts a top view of the blade 10, including the distal end 12, proximal end 14, and elongated body 16 in accordance with an exemplary embodiment of the invention. This figure demonstrates the distal location of the first camera 20 in comparison to the proximal location of the second camera 30 along the elongated body 16 of the blade 10. In an exemplary embodiment of the intubating device 100, the intubating device 100 would be inserted into the oropharynx of the subject with the distal end 12 first. During insertion of the blade 10 into the oropharynx, the top of the blade faces cephalad within the oral cavity respective to the subject. As such, first camera 20 points forward, posteriorly, and distal into the oral cavity whereas the second camera 30 points lateral and proximal within the oral cavity.

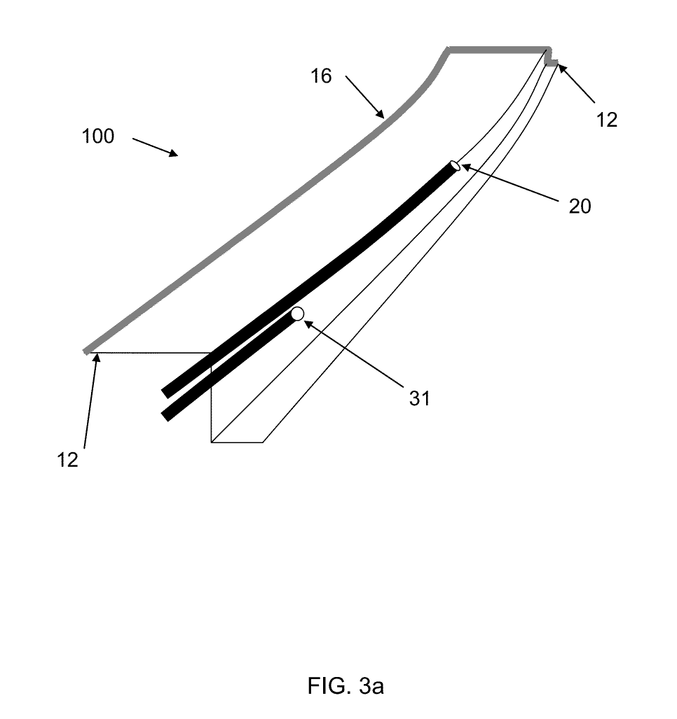

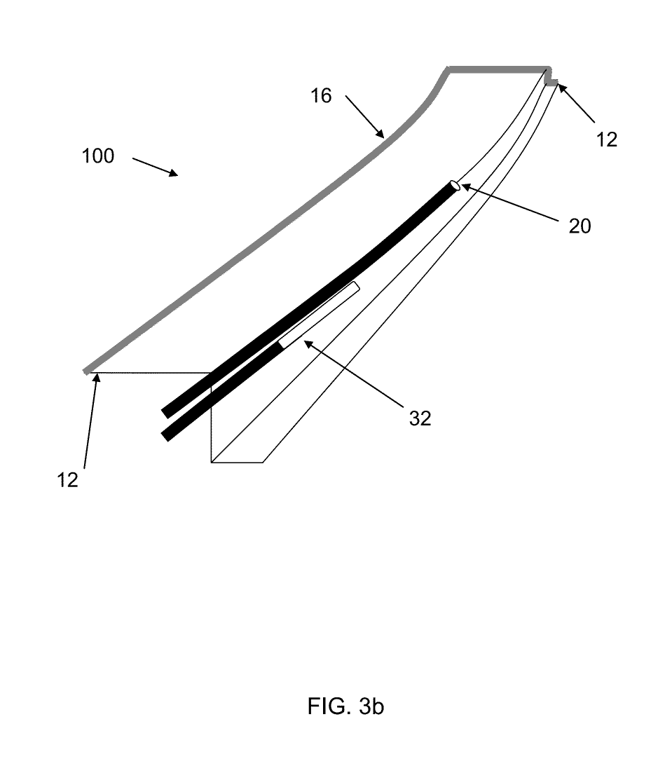

FIGS. 3A and 3B depict a perspective view of the intubating device 100 in accordance with an exemplary embodiment of the invention. The second camera 30 as described in FIG. 2 is subject to adjustable length from the distal end 12 of the blade 10 and camera type in these figures. FIG. 3A depicts the second camera 31 as a single, wide-angle camera. FIG. 3B depicts the second camera 32 as a panoramic camera. In an exemplary embodiment of the intubating device 100, the location and type of second camera would be utilized to optimize view of impinging oropharyngeal structure. Identification and continued view of this allows for the safe passage of styleted endotracheal tube throughout the oropharynx.

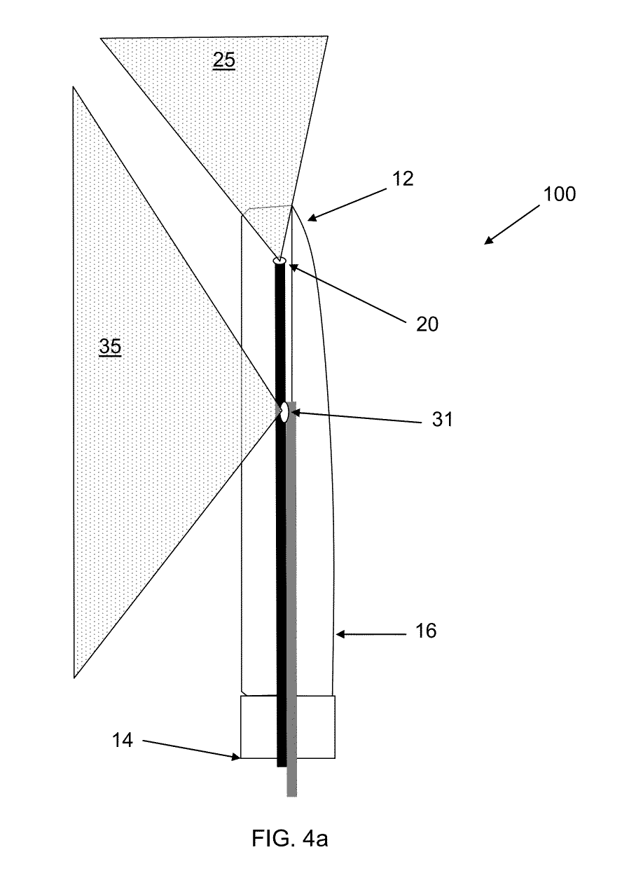

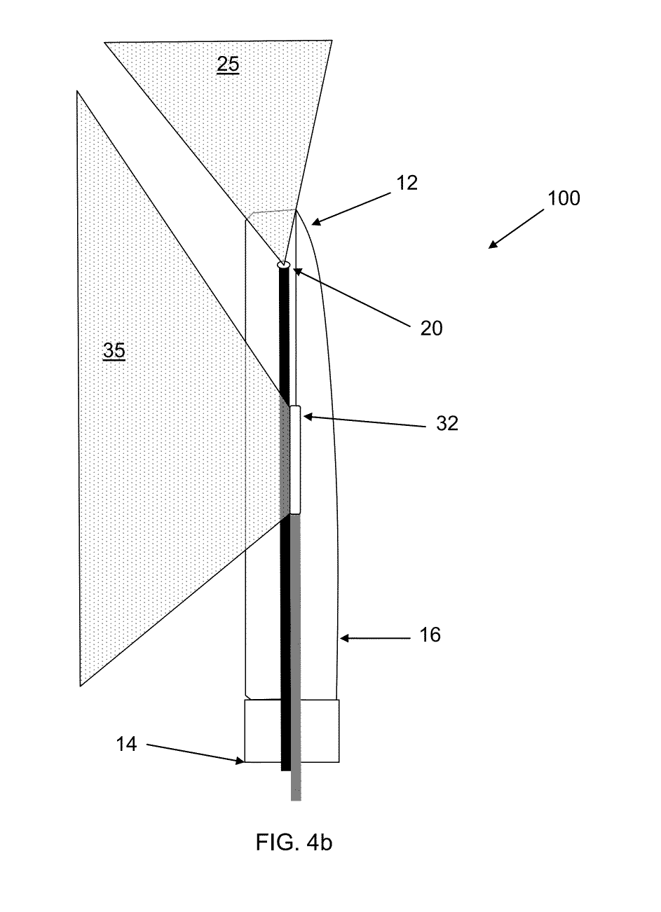

FIGS. 4A and 4B depict a top view of the blade 10 in accordance with an exemplary embodiment of the invention. This figure depicts the field of view 25 that will be visibly displayed from the first camera 20. FIG. 4A further depicts an embodiment of the field of view 35 that will be visibly displayed from a wide-lens second camera 31. FIG. 4A depicts an embodiment of the field of view 35 that will be visibly displayed from a panoramic second camera 32. In an exemplary embodiment, the field of view 25 of the first camera 20 would provide an image in which the glottic structures are visible after the intubating device 100 has been advanced within the oropharynx of the subject. In an exemplary embodiment, the field of view 35 of the second camera 31, 32 would provide an image of the soft pharyngeal structures such as tonsils, palatoglossal arch and palatoglossal arch. After placement of the intubating device 100 into the oropharynx of the subject, the field of view 35 of the second camera 31 in FIG. 4A or the second camera 32 in FIG. 4B, provides vital information of the anatomy of the oropharynx of the subject. This field of view 35 provides users with a simultaneous view of these structures during the advancement of the styleted intubating tube until the point at which it may be adequately visualized within the field of view 25 of the first camera 20. After a user has further advanced a styleted intubating tube within the oropharynx of the subject, the tip of the intubating tube would be visible within this field of view 25 in order to facilitate its proper placement.

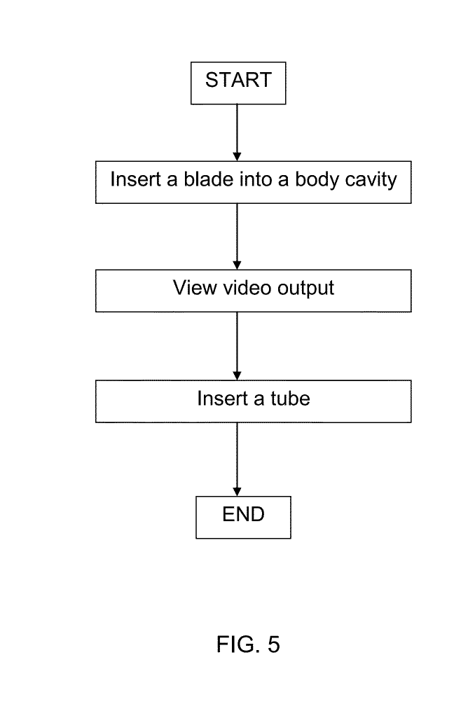

FIG. 5 depicts a method of intubating using a multi-camera intubation device 100 in accordance with an exemplary embodiment of the invention. The method includes the step of inserting a blade 10 into a body cavity, the blade 10 having distal 12 and proximal 14 ends separated by an elongated body 16 with first 20 and second 30 cameras located thereon. The first camera 20 is located a predetermined distance from the distal end 12 of the blade 10 and provides a unidirectional, forward field of view. The second camera 30 is located proximal to the first camera 20 and providing a field of view that is substantially orthogonal to the field of view of the first camera 20. The method also includes the step of viewing, on a display, a video output of the second camera 30 to detect tissue obstructions. The method further includes, guided by the blade, the step of inserting a tube into the body cavity in a manner that minimizes trauma to the tissue obstructions detected by the second camera 30. In exemplary embodiments the tube comprises a rigid stylet.

In an exemplary embodiment of the method of intubation, the user would first place the intubating device 100 in the subject's oral cavity through direct vision. Next, the intubating device 100 would be advanced such that an image of the subject's glottis is visible through the distal camera 20. The styleted intubating tube would then be placed in the subject's mouth via direct vision. The intubating tube should then be advanced towards the subject's glottis until it can no longer be seen under direct visualization. The styleted intubating tube should then be carefully advanced while visualizing its passage through the oropharynx on a display of the second camera 30. The styleted intubating tube should be advanced until its tip is visualized on the display of the first camera 20. The styleted intubating tube should then be advanced through the subject's glottic opening. The stylet should be withdrawn and the intubating tube should be advanced further until the cuff of the intubating tube is past the vocal cords. The intubating tube is then to be held in place while the intubating device 100 is removed from the mouth.

While the invention has been described in connection with preferred embodiments, it will be understood by those of ordinary skill in the art that other variations and modifications of the preferred embodiments described above may be made without departing from the scope of the invention. Other embodiments will be apparent to those of ordinary skill in the art from a consideration of the specification or practice of the invention disclosed herein. The specification and the described examples are considered as exemplary only, with the true scope and spirit of the invention indicated by the following claims.

What is claimed is:

1. A multi-camera intubation device, comprising:
a blade having distal and proximal ends separated by an elongated body;
a first camera located on said elongated body a distance from said distal end of said blade and providing a unidirectional, forward field of view; and
a second camera located on said elongated body a predetermined distance proximal to said first camera and providing a field of view substantially orthogonal to said field of view of said first camera.

2. The multi-camera intubation device of claim 1, wherein said field of view of said second camera is wide-angle.

3. The multi-camera intubation device of claim 1, wherein said field of view of said second camera is panoramic.

4. The multi-camera intubation device of claim 1, wherein said distance of said second camera from said first is adjustable.

5. The multi-camera intubation device of claim 1, further comprising one or more light sources coupled to said blade to provide illumination.

6. The multi-camera intubation device of claim 1, further comprising a handle coupled to said proximal end of said blade to facilitate gripping by an operator.

7. The multi-camera intubation device of claim 1, further comprising a third camera located on said elongated body in the vicinity of said second camera and providing a field of view orthogonal to said field of view of said first camera and opposite of said field of view of said second camera.

8. A multi-camera tracheal intubation device, comprising:
a blade having distal and proximal ends separated by an elongated body forming a radius adapted to conform to a contour of an oropharynx;
a first camera located on said elongated body a predetermined distance from said distal end and providing a unidirectional, forward, laryngeal field of view; and
a second camera located on said elongated body a predetermined distance proximal to said first camera and providing a pharyngeal field of view substantially orthogonal to said field of view of said first camera to visualize a pharyngeal structure.

9. The multi-camera tracheal intubation device of claim 8, wherein said field of view of said second camera is wide-angle.

10. The multi-camera tracheal intubation device of claim 8, wherein said field of view of said second camera is panoramic.

11. The multi-camera tracheal intubation device of claim 8, wherein said distance of said second camera from said first is adjustable.

12. The multi-camera tracheal intubation device of claim 8, further comprising one or more light sources coupled to said blade to provide illumination.

13. The multi-camera tracheal intubation device of claim 8, further comprising a handle coupled to said proximal end of said blade to facilitate gripping by an operator.

14. The multi-camera tracheal intubation device of claim 8, further comprising a third camera located on said elongated body in the vicinity of said second camera and providing a field of view orthogonal to said field of view of said first camera and opposite of said field of view of said second camera.

15. A method of intubating using a multi-camera intubation device, comprising:
inserting a blade into a body cavity, said blade having distal and proximal ends separated by an elongated body with first and second cameras located thereon, said first camera located a predetermined distance from said distal end of said blade and providing a unidirectional, forward field of view, said second camera located proximal to said first camera and providing a field of view substantially orthogonal to said field of view of said first camera;
viewing, on a display, a video output of said second camera to detect tissue obstructions; and
guided by said blade, inserting a tube into said body cavity in a manner that minimizes trauma to said tissue obstructions detected by said second camera.

16. The method of claim 15, wherein said tube comprises a rigid stylete.
